(19) United States
(12) Patent Application Publication
(10) Pub. No.: US 2003/0018243 A1
(43) Pub. Date: Jan. 23, 2003

(54) Selectively plated sensor

(76) Inventors: Thomas J. Gerhardt, Littleton, CO (US); Yassir Abdul-Hafiz, Irvine, CA (US); Eugene E. Mason, La Mirada, CA (US); David R. Tobler, Westminster, CO (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/165,615

(22) Filed: Jun. 7, 2002

Related U.S. Application Data
(63) Continuation of application No. 09/612,139, filed on Jul. 7, 2000, now abandoned.
(60) Provisional application No. 60/143,045, filed on Jul. 7, 1999.

(51) Int. Cl. A61B 5/00

(52) U.S. Cl. 600/322

(57) ABSTRACT

A selectively plated low-noise optical sensor for non-invasive physiological monitoring has an LED emitter that emits light at a known wavelength. The light propagates through a body material and an attenuated signal is received by a photodetector, which produces an electrical signal indicative of the intensity of light energy incident on the detector. The electrical signal is conducted through a plurality of traces to a contact end of the sensor. The contact end allows connection to a connector which communicates the electrical signal to a processor. The emitter and detector are connected to the sensor traces at trace connection pads in the component connection areas. The trace connection pads in the contact area and the connection areas are electroplated with a protective metallic layer. The traces are otherwise covered with a solder mask. In this manner, solderability of the connection pads is enhanced and the traces and connection pads are protected from environmental factors which may cause noise-generating degradation.

### REFERENCE TO RELATED APPLICATION

The present application is a continuation of U.S. patent application Ser. No. 09/612,139, filed on Jul. 7, 2000, entitled “SELECTIVELY PLATED SENSOR,” (the parent application) and claims priority benefit under 35 U.S.C. §120 to the same. The parent application claimed a priority benefit under 35 U.S.C. §119(e) from Provisional Application No. 60/143,045, filed Jul. 7, 1999, entitled “SELECTIVELY PLATED SENSOR.” The present application incorporates each of the foregoing disclosures herein by reference.

### BACKGROUND OF THE INVENTION

1. Field of the Invention

The present invention relates to optical probes used to sense optical energy passed through a medium to determine the characteristics of the medium, and more particularly to optical probes which are selectively plated.

2. Description of the Related Art

Energy is often transmitted through or reflected from a medium to determine characteristics of the medium. For example, in the medical field, instead of extracting material from a patient's body for testing, light or sound energy is transmitted through body tissue and the attenuated transmitted (or reflected) energy may be measured to determine information about the body tissues. This type of non-invasive measurement is comfortable for the patient and can be performed quickly.

Non-invasive physiological monitoring of bodily function is common. For example, during surgery, blood pressure and the body's available supply of oxygen, or the blood oxygen saturation, are often monitored. Oxygen saturation is typically performed with non-invasive techniques by measuring light received after attenuation through a portion of the body, for example a digit such as a finger, earlobe or forehead.

Demand has increased for disposable and reusable optical probes which are suitably constructed to provide low-noise signals for advanced signal processors in order to more accurately determine the characteristics of the medium.

Difficulties arise for advanced signal processing based on signals from optical sensors if the circuit paths conducting the signals degrade or if the sensor is not shielded properly.

### SUMMARY OF THE INVENTION

Accordingly, a need exists for a low-cost, low-noise optical probe which is easy to use, is sufficiently shielded to work with advanced signal processing and whose electrical circuitry does not degrade over time during the manufacturing process or otherwise.

In accordance with one aspect, the present invention includes a probe for use in non-invasive measurement of characteristics of a medium. An emitter transmits optical radiation and a detector is configured to detect the optical radiation transmitted by the emitter and attenuated by the medium. A flexible circuit assembly includes the emitter detector, and a connector tab. The connector tab is adapted to releasably engage a connector. Electrical circuit paths couple the emitter and detector with the connector tab. The electrical circuit paths have component connection areas positioned and adapted to facilitate electrical connection between the paths and the emitter and detector. The paths also have a contact area positioned on the connector tab and adapted to facilitate electrical connection between the paths and the connector. The circuit paths in the contact area and component connection areas are coated with a solderable protective coating. The rest of the circuit paths are coated with non-conductive insulation.

In accordance with another aspect of the present invention, a flexible circuit assembly provides electrical communication between at least one component and a connector. A plurality of electrical circuit paths extend between the connector and the component. At least one contact area is defined along the paths. The component attaches to the paths at one contact area. The circuit paths are covered with a solderable protective coating in the at least one contact area, and the circuit paths are otherwise covered with non-conductive insulation.

In accordance with yet another aspect, the present invention includes a method for making a flexible circuit assembly for a medical sensor. A plurality of electrical circuit paths are formed on at least one side of a flexible substrate. A contact area is defined at a first end of the circuit paths and at least one component connection area is defined at a second end of the circuit paths. An emitter and a detector are provided, the emitter being adapted to transmit optical radiation and the detector being configured to detect the optical radiation transmitted by the emitter. The electrical circuit paths, except for said contact and component connection areas, are coated with insulation. The electrical circuit paths in the contact and component connection areas are coated with a solderable protective coating. The detector is electrically connected to at least one circuit path in the component connection area.

In accordance with a still further aspect of the present invention, an optical probe is provided for non-invasive measurement of charactristics of a medium. An emitter transmits optical radiation and a detector is configured to detect the optical radiation transmitted by the emitter and attenuated by the medium. A flexible circuit assembly includes the emitter, the detector, and a connector tab. The flexible circuit assembly has electrical circuit paths connecting the emitter and detector with the connector tab. The electrical circuit paths are coated with a solderable protective coating comprising a layer of gold.

For purposes of summarizing the invention and the advantages achieved over the prior art, certain objects and advantages of the invention have been described herein above. Of course, it is to be understood that not necessarily all such objects or advantages may be achieved in accordance with any particular embodiment of the invention. All of these embodiments are intended to be within the scope of the invention herein disclosed.

### BRIEF DESCRIPTION OF THE DRAWINGS

FIG. 1 is a perspective view of an optical probe having features in accordance with the present invention.

FIG. 2 shows the optical probe of FIG. 1 at a step in the manufacturing process wherein circuit paths are etched onto a flexible circuit panel.

FIG. 3 is a close-up view of a component end of the optical sensor of FIG. 2.

FIG. 4 is a close-up view of a contact end of the optical sensor of FIG. 2.

FIG. 5 is a flow chart setting forth a method of manufacturing the low noise optical probe of FIG. 1.

FIG. 6 depicts a pair of flexible circuits formed on a flexible substrate during manufacturing of the optical probe of FIG. 1.

FIG. 7 is a perspective view showing a step in the manufacturing process wherein the flex circuits are placed onto a strip of flex circuit shield material.

FIG. 8 depicts the flex circuits of FIG. 7 after being trimmed and including components added during the manufacturing process.

FIG. 9 depicts a step of the manufacturing process wherein medical tape is attached to a group of shielded flex circuit assemblies.

FIG. 10 is a perspective view of completed optical probes having features in accordance with the present invention.

### DETAILED DESCRIPTION OF THE PREFERRED EMBODIMENT

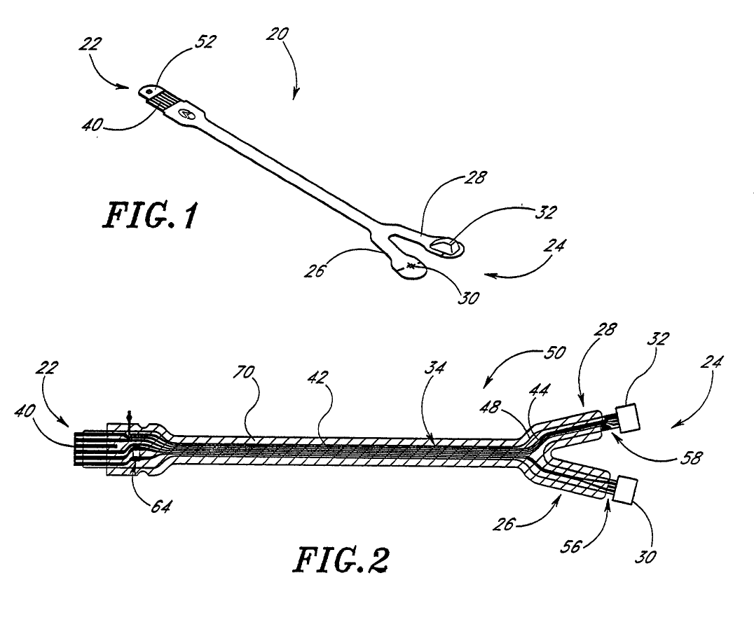

FIG. 1 depicts a low-noise, low-cost optical probe 20 having a contact end 22 and a component end 24. The component end 24 in the embodiment shown is specially adapted for use with neonates and is split into two branches 26, 28 which are oriented in a V-shape. An LED emitter 30 is disposed on a first branch 26 and a detector 32 is disposed on a second branch 28 of the V-shaped component end 24.

In operation, the LED 30 and detector 32 are positioned on opposing sides of tissue to be monitored. The depicted sensor is typically applied to a foot of a neonate. The LED 30 emits light at a known wavelength. The light propagates through the tissue and an attenuated signal is received by the photodetector 32. The photodetector 32 produces an electrical signal indicative of the intensity of light energy incident on the photodetector. The electrical signal is conducted by a circuit path from the detector to a processor which analyzes the signal to determine characteristics of the media through which the light energy has passed. A more detailed discussion of the operation of the LED emitter 30 and the photodetector 32 is provided in assignee's prior patent entitled LOW-NOISE OPTICAL PROBES, U.S. Pat. No. 5,782,757, issued Jul. 21, 1998, which is hereby incorporated by reference in its entirety.

FIG. 2 depicts the LED emitter 30 and the detector 32, shown schematically, each connected to a circuit path 34 which extends to a contact area 40. The circuit path 34 comprises a plurality of flexible conductive traces 42 etched on a signal side 44 of a flexible plastic (preferably polyimide) panel 48. The traces 42 conduct electrical power to the emitter 30 and conduct electrical signals generated by the detector 32. Because the circuit path 34 is intended to be flexible, the conductive traces 42 and the associated panel 48 are collectively referred to as a flex circuit 50.

With reference again to FIG. 1, the contact area 40 is preferably attached to a durable plastic connector tab 52. The combined connector tab 52 and contact area 40 is adapted to be releasably connected to the connector (not shown), which receives electrical signals from the optical probe 20 and in turn conducts the signals to the processor or monitor.

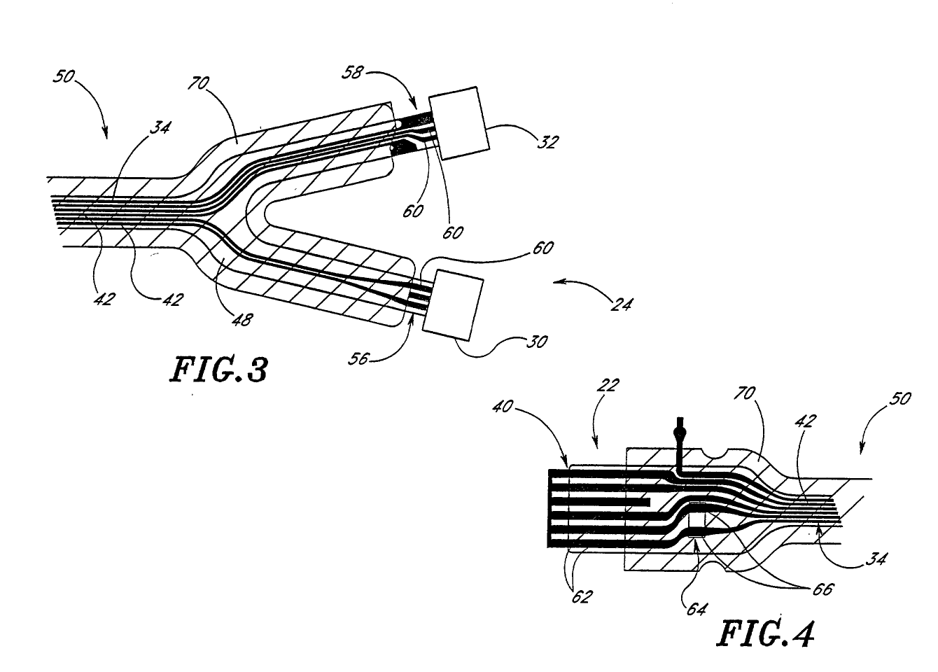

FIG. 3 depicts component connection areas 56, 58 in which the traces 42 are electrically connected to the emitter 30 and detector 32. Preferably, the traces 42 terminate as pads 60 in the connection areas 56, 58. The pads 60 are preferably coated with a protective conductive coating which enhances solderability and protects the pads 60 from environmental factors. This coating will be discussed in more detail below.

With reference to FIG. 4, the traces 42 are widened and become pads 62 in the contact area 40 so as to provide sufficient area to consistently establish electrical attachment with circuit paths within the connector. As with the component connection area pads 60 discussed above, the contact area pads 62 are preferably coated with a protective solderable coating.

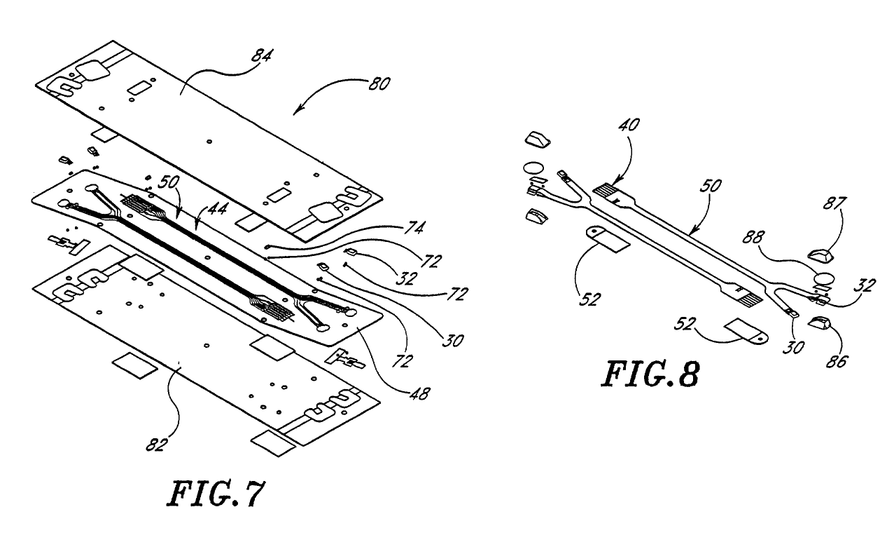

A resistor connection area 64 is defined near the contact area 40 and facilitates electrical contact between two of the traces 42 through a resistor 74 (see FIG. 7). These traces are widened to form resistor connection pads 66 in the resistor connection area 64. As above, the pads 66 are preferably coated with a solderable protective coating in the resistor connection area 64.

Flex circuit traces 42 can be protected from the surrounding environment by a coating of tin that extends substantially the entire length of the flex circuit. It has been discovered, however, that repetitive flexing of the flex circuit tends to create cracks in the protective tin coating. Such failure of the protective coating may expose the traces to environmental factors that may cause or accelerate degradation such as oxidation. Degradation of the traces may result in noise being transmitted along with the signal, which noise can result in inaccurate readings. Accordingly, noise is desirably minimized by the present invention.

As depicted in FIG. 2, except for the connection areas 56, 58, 64 and the contact area 40 described above, the entire flexible circuit 50 is preferably coated with a layer 70 of non-conductive protective insulation. The insulation layer 70 and the protective solderable coatings discussed above work together to enhance solderability and to protect the circuit traces 42 from environmental factors, thus reducing the possibility of noise-generating degradation. To further minimize noise, shielding is preferably provided on either side of the flex circuit.

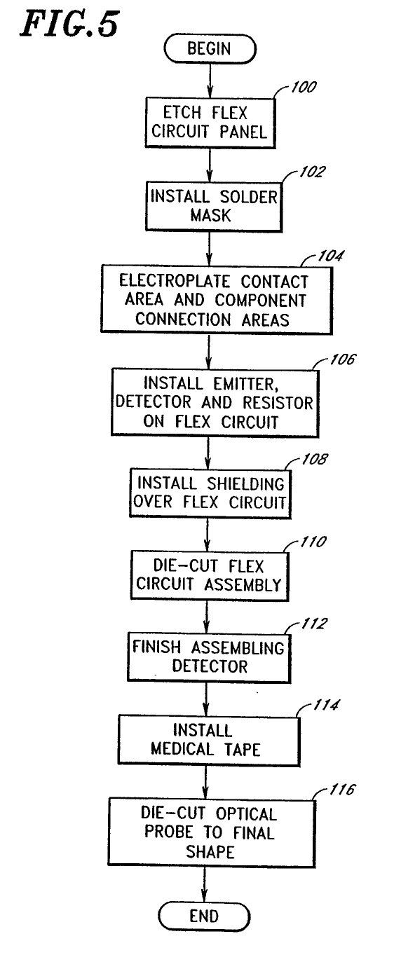

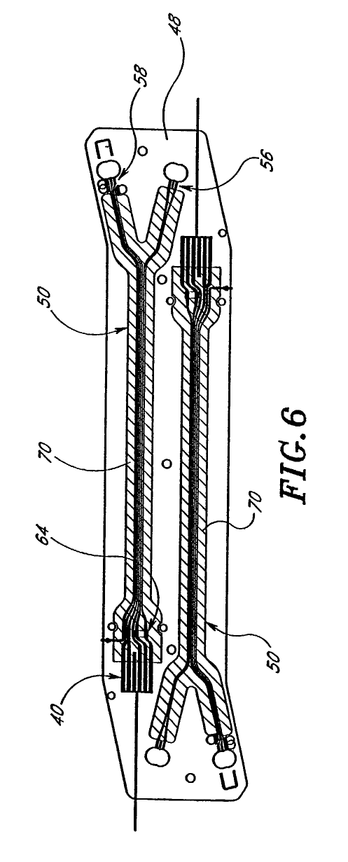

FIG. 5 is a flow chart illustrating general steps in accordance with the present invention to manufacture a first embodiment of the optical probe 20 depicted in FIG. 1. A flex circuit 50 is first created by forming circuit traces 42 on a flex circuit panel 48. In one advantageous embodiment, the flex circuit panel 48 comprises a copper/polyimide or copper/polyester laminate. Most preferably, the latninate is comprised of one-ounce copper (approximately 1.3 mils) over 1 mil of polyimide. Alternatively, any combination of the thicknesses, such as ½ to 1½ ounce copper, or other thicknesses, can also be used. The circuit traces 42 are preferably formed on the panel through etching, as indicated by activity block 100. Alternatively, the circuit traces 42 can be deposited onto the panel using an additive process. As depicted in FIG. 6, preferably a plurality of flex circuits 50 are formed on a single flex panel 48. Such construction enables mass production and makes the flex circuits 50 easier to work with, thus facilitating manufacture.

With reference to FIGS. 2-6, after the flex circuit 50 has been formed on an appropriate substrate material, a layer of insulation 70 is applied over the entire circuit path 34 except for the component connection areas 56, 58, 64 and the contact area 40, as represented in activity block 2 (FIG. 5). Preferably, the insulation comprises a solder mask about 250-750 microinches thick. However, any thickness that provides adequate insulation and allows the flex circuit to bend can be used. Most preferably, the solder mask layer is about 500 microinches thick. The solder mask layer can be formed in any appropriate manner and may use any suitable solder mask material, such as screenable solder mask or dry film photo-imageable solder mask. The layer of solder mask 70 deposited on a flex circuit 50 is preferably cured by exposure to ultraviolet radiation.

After the solder mask 70 has been deposited, the solderable protective coating discussed above is formed on each of the pads 60, 62, 66 in the component connection areas 56, 58, 64 and the contact area 40, as represented in activity block 104 (FIG. 5). The solderable protective coating is preferably a conductive metallic material such as tin or silver. Various combinations, such as a layer of tin applied over a layer of copper, or an alloy of tin and lead applied over a layer of copper, can also be used. Most preferably, the protective coating comprises a layer of hard gold applied over a layer of nickel. The gold-over-nickel protective coating is preferably formed by first electroplating a layer of nickel onto the pads 60, 62, 66 and then electroplating a layer of hard gold over the nickel. The combined gold-over-nickel protective coating preferably has an overall thickness of between about 25 and 50 microinches. It should be appreciated that different materials may require different ranges of thickness.

In an alternative embodiment, prior to or instead of depositing a layer of solder mask, a protective coating of gold or gold-over-nickel is electroplated onto the traces of the flex circuit along substantially the entire length of the flex circuit. The protective coating is preferably about 25-50 microinches thick. The increased ductility of the gold combined with the reduced coating thickness prevents cracking, even under repetitive flexing.

As discussed above, the protective layer is preferably formed by electroplating. However, other processes, such as chemical depositing processes, can appropriately be used.

With reference to FIG. 7, the emitter 30, detector 52 and an identifying resistor 74 are each soldered onto corresponding pads in the appropriate connection areas 56, 58, 64, respectively, of the flex circuits 50, as represented in activity block 106 (FIG. 5). The solder operation is preferably performed through a direct heat reflow of the solder.

In a preferred embodiment, the resistor 74 is connected on either end to the traces that supply power across the LED emitter. The advantages of this parallel connection are explained in detail in assignee's U.S. Pat. No. 5,758,644, entitled MANUAL AND AUTOMATIC PROBE CALIBRATION, which is hereby incorporated by reference in its entirety. In other embodiments, the resistor 74 may be connected to the ground trace on one end and a resistor signal trace at the other end.

Once the appropriate circuit elements are positioned and soldered into place, the flex circuit 50 is enclosed within a shield, as represented in activity block 108 (FIG. 5) and depicted in FIG. 7. As discussed above, multiple flex circuits 50 are preferably processed simultaneously to facilitate efficiency in manufacture. Preferably, the shield 80 comprises a layer of opaque MYLAR™ having one side metallized. However, the shield can be constructed of any flexible plastic film having a conductive coating on at least one side. A bottom shield layer 82 has a metallic side which is preferably positioned against the back side of the flex circuit substrate 48. A conductive pressure sensitive adhesive (PSA) bonds the flex circuit panel 48 to the bottom shielding layer 82. In an alternative embodiment, the back side of the flex circuit panel 48 has a metal coating, such as copper, which provides appropriate shielding. Thus, the bottom shielding layer can be eliminated in an alternative embodiment.

With continued reference to FIG. 7, a top shielding layer 84 is placed to shield the signal side 44 of the flex circuit 50. This second shielding layer 84 preferably comprises the same material as the first shielding layer 82. The top shielding layer 84 covers the flexible circuit and is bonded to the flexible circuit 50 using PSA.

Once the shield 80 is attached, the flex circuit 50 is trimmed as shown in FIG. 8 to remove excess shielding and excess flex paneling. Preferably the flex circuit 50 is trimmed by a die, as represented in activity block 110 (FIG. 5). The connector tab 52 is connected at the contact area 40 and attached with PSA. As represented in activity block 112 and depicted in FIG. 8, components of the photodetector 32, such as a base 86, a cover 87 and a light barrier disk 88 are then assembled as described in the above-referenced application entitled LOW-NOISE OPTICAL PROBE.

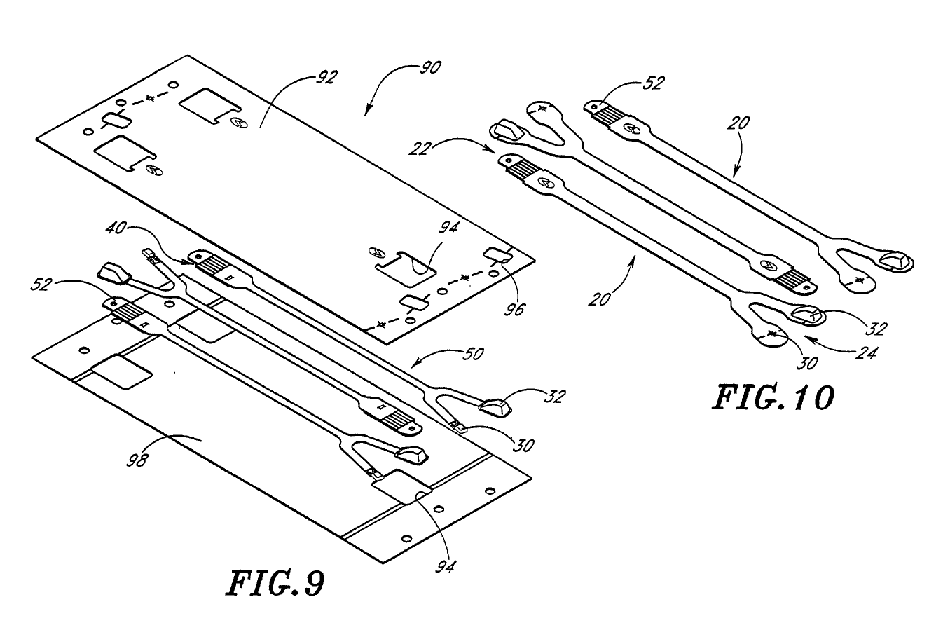

Referring next to FIG. 9, with the shield 80 in place, components 30, 32 assembled and connector tab 52 installed, the flex circuit 90 is ready for an outer covering 50 of medical tape to be applied. This step is referenced in activity block 114 of FIG. 5. The flex circuit assembly 50 is sandwiched between a top and bottom tape layer 92, 98, which are preferably bonded to the flex circuit 50 with PSA. The top and bottom tape layers 92, 98 are preferably configured with openings 94 so that the contact area 40 and connector tab 52 of the flex circuit 50 remains exposed to allow connection of the contacts 62 with the connector. Similarly, holes 96 through the top layer 92 are adapted to receive components of the detector 32 therethrough. Preferably, the bottom tape layer 98 has adhesive portions on one side to facilitate adhesion to the tissue material under test.

The top and bottom tape layers are preferably constructed from a conventional medical tape made from non-woven face material, but any appropriate covering material may be used. As depicted in FIG. 9, a plurality of flex circuits 50 are preferably covered with medical tape at the same time. This facilitates economy in the manufacturing process. Once the tape has been applied, the optical probes 20 are trimmed to a finished state as represented in activity block 116 (FIG. 5) and depicted in FIGS. 1 and 10.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above, but should be determined only by the claims that follow.

What is claimed is:

1. An optical probe for non-invasive measurement of characteristics of a medium, comprising:
an emitter which transmits optical radiation;
a detector configured to detect said optical radiation transmitted by said emitter and attenuated by said medium; and
a flexible circuit assembly including said emitter, said detector, and a connector tab, said connector tab adapted to releasably engage a connector, said flexible circuit assembly having electrical circuit paths coupling said emitter and said detector with said connector tab, said electrical circuit paths having component connection areas positioned and adapted to facilitate electrical connection between said paths and said emitter and detector, and a contact area defined on the connector tab and adapted to facilitate electrical connection between said paths and said connector;
wherein the circuit paths in the contact area and component connection areas are coated with a solderable protective coating, and the rest of said circuit paths are coated with non-conductive insulation.

2. The optical probe of claim 1, wherein the circuit paths comprise copper cladding formed on a single side of a polyimide film.

3. The optical probe of claim 1, wherein the insulation comprises a layer of solder mask.

4. The optical probe of claim 3, wherein the solder mask is about 250-750 microinches thick.

5. The optical probe of claim 1, wherein the protective coating comprises a metallic layer.

6. The optical probe of claim 5, wherein the protective coating comprises a layer of gold.

7. The optical probe of claim 6, wherein the layer of gold is overlaid onto a layer of nickel.

8. The optical probe of claim 7, wherein the protective coating is about 25-50 microinches thick.

9. The optical probe of claim 5, wherein the protective coating comprises material chosen from the group consisting of tin, tin over copper, tin/lead alloy over copper, silver and gold.

10. The optical probe of claim 5, including a resistor and a resistor component connection area, the resistor component connection area positioned and adapted to facilitate electrical connection between a first and a second of said electrical circuit paths via said resistor.

11. A flexible circuit assembly for providing electrical communication between at least one component and a connector, comprising a plurality of electrical circuit paths extending between the connector and the component, at least one contact area being defined along the paths, and the component attaches to the paths at one contact area, the circuit paths being covered with a solderable protective coating in the at least one contact area and the circuit paths being otherwise covered with non-conductive insulation.

12. The flexible circuit assembly of claim 11, wherein the non-conductive insulation comprises a coating of solder mask about 250-750 microinches thick.

13. The flexible circuit assembly of claim 11, wherein the electrical circuit paths in the at least one contact area are coated with a conductive metallic material.

14. The flexible circuit assembly of claim 13, wherein the electrical circuit paths in the at least one contact area are coated with nickel overlaid by gold.

15. The flexible circuit assembly of claim 11, wherein the electrical circuit paths include a resistor contact area defined between a first and second circuit path, and said first and second circuit paths are adapted to receive a resistor extending between them.

16. The flexible circuit assembly of claim 15, wherein the first and second circuit paths are coated with a metallic protective layer in the resistor contact area.

17. The flexible circuit assembly of claim 11, including an emitter component and a detector component, said emitter component adapted to transmit optical radiation and said detector component configured to detect said optical radiation transmitted by said emitter and attenuated by a medium disposed between the emitter and detector.

18. A method for making a flexible circuit assembly for a medical sensor, comprising the steps of:
forming a plurality of electrical circuit paths on at least one side of a flexible substrate;
defining a contact area at a first end of the circuit paths and at least one component connection area at a second end of the circuit paths;
providing an emitter adapted to transmit optical radiation;
providing a detector configured to detect said optical radiation transmitted by said emitter;
coating the electrical circuit paths except for said contact and component connection areas with insulation;
coating the electrical circuit paths in said contact and component connection areas with a solderable protective coating; and
electrically connecting the detector to at least one circuit path in the component connection area.

19. The method of claim 18, wherein the flexible substrate is a polyimide film between about 0.75 and 1.25 mil thick and the electrical circuit paths are formed of copper cladding of between about ½-1½ oz.

20. The method of claim 18, wherein the insulation is applied prior to applying the protective coating.

21. The method of claim 18, wherein the protective coating is formed by first depositing a layer of nickel and subsequently depositing a layer of gold over the layer of nickel.

22. The method of claim 21, wherein the layers of gold and nickel are deposited by electroplating.

23. The method of claim 21, wherein the insulation comprises a layer of solder mask deposited on the film and over the circuit paths.

24. The method of claim 23, additionally comprising exposing the flex circuit to ultraviolet radiation to cure the solder mask.

25. The method of claim 23, wherein the solder mask comprises screenable solder mask.

26. The method of claim 23 wherein the solder mask comprises dry film photo-imageable solder mask.

27. The method of claim 18, additionally comprising defining a resistor component connection area, the resistor component connection area including portions of a first and a second circuit path.

28. The method of claim 27, additionally comprising attaching a resistor to the first and second circuit paths in the resistor component connection area after the protective coating has been applied.

29. An optical probe for non-invasive measurement of characteristics of a medium, comprising:
an emitter which transmits optical radiation;
a detector configured to detect said optical radiation transmitted by said emitter and attenuated by said medium; and
a flexible circuit assembly including said emitter, said detector, and a connector tab, said flexible circuit assembly having electrical circuit paths connecting said emitter and said detector with said connector tab, said electrical circuit paths being coated with a solderable protective coating comprising a layer of gold.

30. The optical probe of claim 29, wherein the protective coating comprises a layer of nickel overlaid by said layer of gold.

31. The optical probe of claim 29, wherein the protective coating is about 25-50 microinches thick.
